(19) United States
(12) Patent Application Publication
(10) Pub. No.: US 2002/0045566 A1
(43) Pub. Date: Apr. 18, 2002

(54) Selective maxi-K potassium channel openers functional under conditions of high intracellular calcium concentration, methods and uses thereof

(76) Inventors: Valentin K. Gribkoff, Wallingford, CT (US); Debra J. Post-Munson, Southington, CT (US); Sarita W. Yeola, Cheshire, CT (US); Christopher G. Boissard, Northford, CT (US); Piyasena Hewawasam, Middletown, CT (US); John E. Starrett, Middletown, CT (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/975,881

(22) Filed: Oct. 12, 2001

Related U.S. Application Data
(60) Provisional application No. 60/240,146, filed on Oct. 13, 2000.

(51) Int. Cl. A61K 31/00

(52) U.S. Cl. 514/1

(57) ABSTRACT

The present invention describes calcium sensitive and selective maxi-K potassium channel opener/activator compounds that function to open maxi-K channels under conditions of high intracellular calcium concentrations, and which do not significantly affect the opening of maxi-K channel proteins under conditions of low or physiologically normal intracellular calcium concentrations. Methods of assaying for and using such compounds are also provided. According to the invention, whole cell voltage patch-clamp studies newly demonstrated that the ability of opener compounds, e.g., fluoro-oxindoles and chloro-oxindoles, to open maxi-K channels was sensitive to the intracellular Ca²⁺ concentration ([Ca²⁺])ᵢ, i.e., more channels opened at more negative potentials. Particular fluoro-oxindole and chloro-oxindole compounds produced significant increases in whole-cell maxi-K potassium channel-mediated outward currents only in cells having higher [Ca²⁺]ᵢ, compared with effects in lower [Ca²⁺]ᵢ. Such compounds provide Ca²⁺-sensitive and selective openers of maxi-K channels which show maximum effectiveness under conditions of increased [Ca²⁺]ᵢ and, as such, provide treatments for diseases and disorders in which cells undergo, or are subject to, traumatic stress due to high internal calcium levels, such as stroke.

### CROSS-REFERENCE TO RELATED APPLICATIONS

This is a nonprovisional application which claims benefit of provisional application U.S. Ser. No. 60/60/240,146 filed Oct. 13, 2000.

### FIELD OF THE INVENTION

The invention relates to the identification of modulators, particularly openers or activators, of large conductance potassium channels (maxi-K⁺, maxi-K, or BK potassium channels) which are highly sensitive to intracellular calcium concentration and have been newly discovered to selectively open these channels under conditions of high intracellular calcium. Such calcium-sensitive openers may be especially effective in the treatment of diseases and disorders associated with conditions of elevated intracellular calcium concentration, particularly, neurological or neurodegenerative pathologies, diseases and disorders, such as ischemic stroke.

### BACKGROUND OF THE INVENTION

Large-conductance calcium (Ca²⁺)-activated potassium (called maxi-K⁺, maxi-K, or BK) channels are found in most tissues, including the brain. Maxi-K channels are unique due to their large conductance and their reliance on the presence of Ca²⁺ and membrane potential for activation. The channels open naturally in response to increase in levels of intracellular Ca²⁺, ([Ca²⁺]ᵢ), and/or membrane depolarization, thus permitting potassium (K⁺) efflux from cells and regulation of cell membrane potential. This activity serves as an endogenous feedback mechanism whereby cells return to less excitable, more hyperpolarized potential, thereby limiting further voltage-dependent Ca²⁺ entry into cells.

Mammalian maxi-K channels are seven-membrane domain-containing proteins, with a large carboxy-terminal region that is likely involved in the binding of Ca²⁺ [M. Schreiber et al., 1999, Nat. Neurosci., 2:416-421; P. Meera et al., 1997, Proc. Natl. Acad. Sci. USA, 94:14066-14071; V. K. Gribkoff et al., 1997, Advances in Pharmacology, 37:319-347]. The maxi-K channels expressed in human neurons and other tissues include the hSlo1 gene family member [S. I. Dworetzky et al., 1994, Brain Res. Mol. Brain Res. 27:189-193; D. P. McCobb et al., 1995, Am. J Physiol., 269:H767-H777]. Maxi-K channels in neurons exhibit significant phenotype variation as a result of alternative splicing [J. Tseng-Crank et al., 1994, Neuron, 13:1315-1330], beta subunit assembly [S. I. Dworetzky et al., 1996, J. Neurosci., 16:4543-4550 and R. Brenner et al., 2000, J. Biol. Chem., 275:6453-6461] and possible co-assembly with a closely related family member, hSlo2 or Slack [W. Joiner et al., 1998, Nat. Neurosci., 1:462-469]. In spite of this variation, all maxi-K channels are voltage- and Ca²⁺-dependent; therefore, their activation threshold is reached only during periods of significant depolarization and/or when Ca²⁺ levels inside neurons are high [O. McManus, 1991, J Bioenerg. Biomembr., 23:537-560 and K. T. Wann and C. D. Richards, 1994, Eur. J. Neurosci,. 6:607-617]. Consequently, pharmacological blockade of these channels has very modest effects on normal neuronal function [K. N. Juhng et al., 1999, Epilepsy Res., 34:177-186]. However, when these channels are open, they are effective regulators of cell membrane potential.

Stroke is a well known example of a neurological condition that causes death and long-term disability to millions of individuals worldwide. Indeed, in the United States alone, more than 700,000 individuals are afflicted by stroke every year [G. R. Williams et al., 1999, Stroke, 30:2523-2528]. Currently, only a single form of therapy, namely, thrombolysis, has been shown to be effective in improving outcome of acute stroke in a limited patient population [M. Fisher, 1999, J. Thromb. Thrombolysis, 7:165-169].

Acute ischemic stroke, the most common form of the disease, produces a core area of severely damaged tissue, distal to the occluded vessel, and surrounded by a penumbra of tissue at risk of death due to its proximity to the core and to low vascular perfusion [G. Schlaug et al., 1999, Neurology, 53:1528-1537]. Ischemic neurons in the penumbra die as a result of a neurotoxic biochemical cascade initiated by lowered energy availability, excessive excitatory amino acid release, elevated intracellular calcium and neuronal hyperexcitability [U. Dirnagl et al., 1999, Trends Neurosci., 22:391-397].

Neuroprotective compounds, which are designed to afford a measure of protection to neuronal cells at risk following vessel occlusion, have repeatedly failed in clinical trials, despite promising preclinical supporting data [K. K. Jain, 2000, Exp. Opin. Invest Drugs, 9:695-711]. Lack of success using neuroprotective compounds in human clinical trials has been attributed to the presence of side-effects, thereby limiting their utility or dose. Other agents that have been tested to completion have failed to demonstrate efficacy [J. De Keyser et al., 1999, Trends Neurosci., 22:535-540].

Since maxi-K channels play a critical regulatory role in cell functioning, these channels constitute an important therapeutic target, particularly, in neuronal cells affected during stroke. Modulation of these channels could provide a therapeutic option for protecting cells exposed to conditions of hyperexcitability or pathogenic levels of Ca²⁺, particularly neuronal cells, and/or cells of neuronal origin. Openers of maxi-K channels have been described, but these openers have been found to lack sufficient potency and specificity to be useful as therapeutic agents [V. K. Gribkoff et al., 1996, Mol. Pharmacol., 50:206-217; J. E. Starrett et al., 1996, Curr. Pharm. Design, 2:413-428].

Needed in the art are new modulators, particularly, openers or activators, of maxi-K channels that exhibit selective action on targeted maxi-K channel proteins in cells under particular conditions, such as elevated internal cellular calcium concentration. Such opener compounds can be designed generally for the treatment of diseases in which the intracellular environment is one of high calcium concentration and in which maxi-K channels are targeted by the opener compounds. More particularly, such opener compounds can be designed for the treatment of neuronal pathologies and diseases, such as stroke, particularly, ischemic stroke, on the basis of physiological processes that occur in cells that are destroyed by neurotoxicity during stroke.

An understanding of the conditions that occur in cells, notably ischemic cells, in which maxi-K channels are active, and the discovery and design of maxi-K openers or agonists, comprising drugs, compounds, small molecules, therapeutic agents, and the like, which are condition-dependent and which exert their effects under such conditions to activate targets in cells at risk, is also required in the art and provided to the art by the present invention.

The present invention provides effective and selective opener compounds, and methods of use thereof. These compounds are designed to be condition-dependent in their actions on maxi-K channels such that the openers work effectively under high intracellular calcium conditions, for example, those of the ischemic cell, thereby affording ischemic protection, while having virtually no effect on normal cells, and/or cells which do not have high such intracellular calcium conditions. By contrast, other opener compounds have been shown to lack sensitivity to intracellular calcium concentration and thus their actions are independent of the concentration of intracellular calcium

As described herein, the present invention defines new calcium-dependent openers, or calcium co-agonists, of maxi-K channels, which exhibit potent and specific opening function of these channels at high levels of intracellular calcium, but which have little effect on these channels in cells having low intracellular calcium levels.

### SUMMARY OF THE INVENTION

The present invention provides novel maxi-K channel opener or activator compounds that increase the open probability of, and augment the function of, mammalian maxi-K channels. Such openers are sensitive to the intracellular calcium concentration of cells, and are demonstrated to be most effective under conditions of increased intracellular calcium concentrations, e.g., micromolar range, while being minimally effective, or not at all effective, under normal, e.g., physiological, to low intracellular calcium concentrations. In a particular aspect, the present invention relates to novel neuroprotective agents in the form of maxi-K channel opener compounds that function under conditions of high intracellular calcium levels, such as those present in neurons at risk during neurodegenerative diseases, such as stroke.

It is also an object of the present invention to provide methods and compounds to modulate a target, i.e., maxi-K channel proteins, that comprise an endogenous system for limiting neuronal excitability and Ca²⁺ entry in cells of the nervous system. According to this invention, novel compounds are designed to open maxi-K channels when intracellular Ca²⁺ levels are higher than normal, e.g., in the micromolar range, such as during acute stroke and related conditions.

It is another object of the present invention to provide methods and compounds that specifically target ischemic neurons to afford ischemic protection by modulating maxi-K channel proteins, particularly, human maxi-K channels, in such neurons by augmenting the function of the maxi-K channels, thereby allowing for condition-dependent activation of a pharmaceutical target and limiting the actions of the compounds to ischemic cells having high levels of intracellular Ca²⁺ compared with other cells, and non-ischemic cells.

It is yet another object of the present invention to provide pharmaceutical compositions comprising the selective maxi-K potassium channel openers of the present invention, wherein the compositions further comprise a pharmaceutically acceptable carrier, excipient, or diluent.

Yet another object of the present invention is to provide assays and methods for screening for, identifying, or detecting maxi-K channel modulators, agents, or compounds which function selectively under conditions of high intracellular calcium concentrations to open maxi-K potassium channels, e.g., to increase K⁺ efflux, and reduce voltage-dependent Ca²⁺ influx. Preferred are opener compounds that affect maxi-K potassium channel proteins and which function to selectively exert their activity on maxi-K channel proteins under conditions of high intracellular calcium concentration, but which do not exert their activity on maxi-K channel proteins under conditions of normal or low intracellular calcium concentration. Such screening methods can include assessments of the ability of such modulators, agents, or compounds to target and effect maxi-K channels under conditions of high intracellular Ca²⁺ levels, compared with their ability to target and effect maxi-K channels under conditions of normal or low intracellular Ca²⁺ levels.

It is a further object of the present invention to provide novel opener compounds and maxi-K modulators as maxi-K channel agonists or activators, such as particularly obtained from the screening methods described.

It is a further object of the present invention to provide methods for the treatment or prevention of diseases in mammals, particularly humans, characterized by affected cells having high levels of intracellular calcium in which maxi-K channel proteins are specifically targeted as a result of the high intracellular calcium concentration of the affected cells. Examples of such diseases include, but are not limited to, stroke, global cerebral ischemia, traumatic brain injury, Parkinson's disease, epilepsy, migraine and chronic neurodegenerative disorders such as Alzheimer's disease.

It is yet a further particular object of the present invention to provide methods for the treatment or prevention of neurological diseases, particularly stroke, and more particularly, acute ischemic stroke, in mammals, preferably humans, employing openers of maxi-K channels having the selective ability to target and activate maxi-K channels in cells having high levels of intracellular Ca²⁺ (e.g., ischemic neurons), while not significantly targeting and activating non-ischemic or normal neurons.

It is another object of the present invention to provide new classes, and exemplary members thereof, of Ca²⁺-sensitive and selective maxi-K channel opener compounds.

It is yet another object of the present invention to provide chloro-oxindole compounds newly-discovered to be Ca²⁺-sensitive and selective maxi-K channel openers as further described herein.

Further objects, features and advantages of the present invention will be better understood upon a reading of the detailed description of the invention when considered in connection with the accompanying figures/drawings.

### BRIEF DESCRIPTION OF THE FIGURES

FIGS. 1A and 1B demonstrate the effects of the maxi-K potassium channel opener compound, BMS-204352, a 3-substituted oxindole derivative, on peak whole cell currents in HEK-293 cells transfected with an hSlo α-subunit in cells perfused with medium containing a high level (1.0 μM) of intracellular Ca²⁺. FIG. 1A shows a group current-voltage relationship (mean±SE; n=5 except wash, n=3). FIG. 1B presents the dose response for the data in FIG. 1A. Current amplitude in drug was measured at +30 mV and expressed as a percent of control current measured at +30 mV.

FIGS. 2A-2C demonstrate the effects of intracellular calcium concentration [Ca²⁺]ᵢ and compound BMS-204352 (5 μM) on whole-cell currents in HEK-293 cells transfected with an hSlo α-subunit. FIG. 2A shows the I-V relationships of whole-cell outward currents under conditions of varying internal [Ca²⁺]_{free}. (i.), (open symbols), represent control conditions and (ii.), (closed symbols), represent 5 μM BMSCT-204352 (mean±SE; each data point n=3 to 20; data from FIGS. 1A and 1B are included in the 1 μM Ca²⁺ group). FIG. 2B presents the data in FIG. 2A with current presented as a ratio (current in drug over control current) versus voltage. FIG. 2C shows representative currents from an experiment with 1 μM [Ca²⁺]ᵢ. As in FIG. 2A, (i.) represents control and (ii.) is carried out in the presence of 5 μM of the BMS-204352 compound.

FIGS. 3A and 3B show the effects of various [Ca²⁺]ᵢ and the opener compound BMS-225113 (10 μM) on outward potassium currents in HEK-293 cells transfected with a cloned hSlo α-subunit using the whole-cell patch-clamp technique. The holding potential was −80 mV for [Ca²⁺]ᵢ=250 nM and 1 μM, and −100 mV for [Ca²⁺]ᵢ=2.5 μM. A series of 10 mV depolarizing voltage steps appropriate to the [Ca²⁺]ᵢ was applied. Increasing [Ca²⁺]ᵢ from 250 nM to 1.0 μM, or to 2.5 μM, shifted the peak current-voltage relationships to the left, thus indicating an apparent leftward shift in V_{½}. In FIG. 3A, compound BMS-225113 (10 μM) produced greater increases in maxi-K currents under conditions of higher [Ca²⁺]ᵢ. In FIG. 3B, the data in FIG. 3A are presented as the ratio of current in drug over control current versus voltage. (Each data point represents the mean±SE of 3 to 9 cells).

FIGS. 4A-4F present the results of in vivo studies in which the opener compound BMS-204352 was administered in several rat models relevant to stroke. FIG. 4A shows modulation of the electrically-stimulated release of [³H]glutamate from rat hippocampal tissue wedges in vitro by the BMS-204352 compound. The compound modestly but potently inhibited the release of radiolabeled glutamate. ***p<0.005, **p<0.01, *p<0.05.3. FIG. 4B shows that the administration of BMS-204352 (IV bolus) to anesthetized rats in vivo, measured at 2 hours after drug injection, resulted in small but significant reductions in the field potential recorded in area CA1, produced by stimulation of the contralateral commissural fiber system in area CA3. A broad range of doses, 50 ng/kg to 1 mg/kg, were effective in this model. **p<0.01, *p<0.05. Inset shows an example of the evoked potential prior to (i.) and following (2 hours; ii.) 100 ng/kg of BMS-204352 administration. These effects were always long-lasting, and persisted for the duration of the longest experiments (4-5 hr). FIG. 4C shows the reduction of cortical infarct volume as measured using magnetic resonance imaging (MRI) by Compound 1 administered 2 hours following permanent unilateral MCA occlusion in the SHR rat. These results, observed with MRI, were confirmed with histological techniques at 24 hours after occlusion onset. *p<0.05. FIG. 4D shows a comparison of the effects of racemic Compound 1 and BMS-204352 (0.3 mg/kg IV) on cortical infarct volume in the Wistar normotensive rat (combined model with permanent unilateral MCA occlusion, permanent ipsilateral CCA occlusion, transient contralateral CCA occlusion; PUM,PIC,TCC model, BMS-204352 administered 2 hours after MCA occlusion onset). **p<0.01. FIG. 4E shows the dose-response relationship for BMS-204352, administered 2 hours after MCA occlusion onset, in the Wistar normotensive rat PUM,PIC,TCC model, demonstrating a similar effective dose-response relationship to that observed with synaptic modulation in FIG. 4B. The mechanism of reversal of efficacy at doses at or above 3 mg/kg in stroke and evoked potential models is not known. **p<0.01, *p<0.05. FIG. 4F shows a comparison of the effects of dosing BMS-204352 (1 mg/kg IV) at 1 or 2 hours post-occlusion onset on the reduction in cortical infarct volume in the Normotensive Wistar rat PUM,PIC,TCC model. At these 2 post occlusion time points, the effects of the maxi-K channel opener are not statistically distinguishable. **p<0.01, *p<0.05. All data were collected at 24 hours after occlusion onset.

FIGS. 5A-5D depict the effects of intracellular Ca²⁺ concentration ([Ca²⁺]ᵢ) and the use the BMS-A compound or the known benzimidazolone compound NS-1619 on whole-cell currents in HEK-293 cells expressing a human brain hSlo α-subunit. FIG. 5A shows maxi-K current-voltage (I-V) relationships for control (open symbols) and BMS-A (1 μM) (closed symbols) under two conditions of [Ca²⁺]ᵢ (i.e., [Ca²⁺]ᵢ of 50 nM and [Ca²⁺]ᵢ of 2.5 μM). FIG. 5B shows the I-V data with current presented as a ratio (current in drug over control current) versus voltage for the data in FIG. 5A. Each data point represents the mean±standard error (SE) of 4-8 cells FIG. 5C shows maxi-K I-V relationships for experiments with the known benzimidazolone NS-1619 under conditions of [Ca²⁺]ᵢ at 50 nM and at 2.5 μM. The control is represented by open symbols and the drug is shown by closed symbols. FIG. 5D shows the I-V data of FIG. 5C with current presented as a ratio versus voltage for the data in FIG. 5C.. Each data point represents the mean±SE of 9-11 cells. Note that BMS-A (FIGS. 5A, 5B) actually reduces maxi-K channel current in low (eg. 50 μM) [Ca²⁺]ᵢ, while NS-1619 produces a similar level of current increase at both [Ca²⁺]ᵢ.

### DETAILED DESCRIPTION OF THE INVENTION

The present invention provides novel maxi-K channel opener compounds that increase the open probability of, and augment the function of, mammalian maxi-K potassium channels, particularly, human maxi-K channels. Such openers have been newly characterized as being sensitive to the intracellular calcium concentration of cells, and are demonstrated to be most effective under conditions of increased intracellular calcium concentrations, e.g., micromolar range, while being minimally effective or not at all effective under normal, e.g., physiological, to low intracellular calcium concentrations. The openers described herein thus require that the intracellular calcium concentration be above that of the resting intracellular calcium concentration in order to exert their activity on cellular maxi-K potassium channels.

In a particular aspect, the present invention relates to novel neuroprotective agents in the form of maxi-K channel opener compounds that function under conditions of high intracellular calcium levels, such as those present in neurons at risk during neurodegenerative diseases, such as stroke.

Maxi-K ion channels are proteins that react to substantial increases in intracellular Ca²⁺ and membrane depolarization by markedly increasing potassium (K+) efflux, rapidly hyperpolarizing the membrane and reducing further voltage-dependent Ca²⁺ influx (V. K. Gribkoff et al., 1997, Adv. PharmacoL, 37:319-348). This invention relates to the discovery that certain maxi-K channel opener compounds can selectively exert their action on maxi-K channels in cells having a high intracellular calcium concentration, while exerting minimal action, or no action, on maxi-K channels in cells having a normal, moderate, or low intracellular calcium concentration.

Thus, the novel opener compounds (“openers” or “maxi-K openers”) according to this invention are extremely sensitive to the intracellular calcium concentration of cells, and are demonstrated to be most effective under conditions of increased calcium, while being minimally effective, or not at all effective, under normal to low physiological intracellular calcium concentrations. This is of particular importance in the case of diseases, conditions, or disorders in which increases or accumulations of intracellular calcium cause traumatic stress to cells and/or are related to pathology, cell toxicity, apoptosis, or death. Acute ischemic stroke is a particular disease in which the accumulation of intracellular Ca²⁺ appears to be major proximal cause of the eventual death of cells in the ischemic penumbra (D. W. Choi, 1995, Trends Neurosci., 18:58-60; T. Kristian and B. K. Siesjo, 1998, Stroke, 29:705-718).

The maxi-K openers of this invention are designed to specifically target cells with high intracellular calcium levels, such as ischemic neurons. Thus, because the activity of the compounds newly provided by this invention is limited and selective for those cells having high concentrations of internal calcium, these compounds are provided by the invention to significantly reduce side-effects, such as lowering blood pressure. In addition, the compounds and their uses according to the invention leave virtually unaffected those cells having normal or low internal calcium levels. As guidance, non-limiting examples of high levels of intracellular calcium are typically considered to be in the high nanomolar (e.g., greater than about 250 or 300 nM) to micromolar range (e.g., about 1 to 10 μM); normal or physiological levels of intracellular calcium are typically considered to be in the range of about 50 nM to 250 nM; and low levels of intracellular calcium are typically considered to be in the range of about 5 to 50 nM.

The compounds of the present invention have been designed so that Ca²⁺ sensitivity is associated with their action on maxi-K channels, thus further limiting their influence to cells that are at risk in disease, and also, in the case of neurodegenerative diseases such as stroke, affording ischemic protection for those cells having higher than normal levels of Ca²⁺ during acute stroke and related conditions. By building into the activity of maxi-K channel opener compounds a uniquely high level of Ca²⁺ sensitivity, the present invention offers more effective neuroprotectants with minimal impact on non-ischemic cells. For neurological disorders and diseases, this greatly decreases drug action on maxi-K channels in neurons and other cells not exposed to potentially pathologic levels of intracellular Ca²⁺.

Accordingly, the compounds and methods described herein are provided so as to greatly decrease drug action on maxi-K channels in normal or unaffected neurons, as well as in other cells that are not exposed to potentially pathologic or lethal levels of intracellular Ca²⁺. By creating opener drugs/compounds which are uniquely sensitive to high levels of intracellular Ca²⁺, there should be little disruption in Ca²⁺ and K⁺ regulation in non-ischemic cells, or other types of cells, which are not characterized by having high intracellular Ca²⁺ levels. In addition, such opener drugs/compounds provide a neuroprotective role for maxi-K channel openers in the amelioration of ischemic stroke.

Compounds suitable for use as maxi-K channel openers are those which are active in the presence of high intracellular calcium concentrations, while having little to no significant activity on the opening of maxi-K channels under conditions of low or normal physiological intracellular calcium concentrations. For employment as neuroprotective agents, the compounds are preferably brain penetrable.

One class of compounds having selective function on cells having high intracellular calcium concentrations encompasses the 3phenyl substituted oxindole derivatives, as described in U.S. Pat. Nos. 5,565,483 and 5,602,169 to P. Hewawasam et al., the contents of which are incorporated by reference herein. Fluoro-oxindole compounds are within the above-described class and are capable of acting selectively as maxi-K channel openers on cells having high intracellular calcium concentration, and not acting to an appreciable extent to open maxi-K potassium channels in cells having normal, moderate or low intracellular calcium concentration.

One member of the fluoro-oxindoles is the maxi-K opener compound (3S)-(+)-(5-Chloro-2-methoxyphenyl)-1,3-dihydro-3-fluoro-6-(trifluoromethyl)-2H-indol-2-one, or BMS-204352, which has been newly determined according to this invention to be a selective and effective opener of a human brain maxi-K channel α-subunit, hSlo, (S. Dworetzky et al., 1994, Brain Res. Mol. Brain Res., 27:189-193) expressed in human embryonic kidney (HEK-293) cells under conditions of elevated or high intracellular calcium concentration.

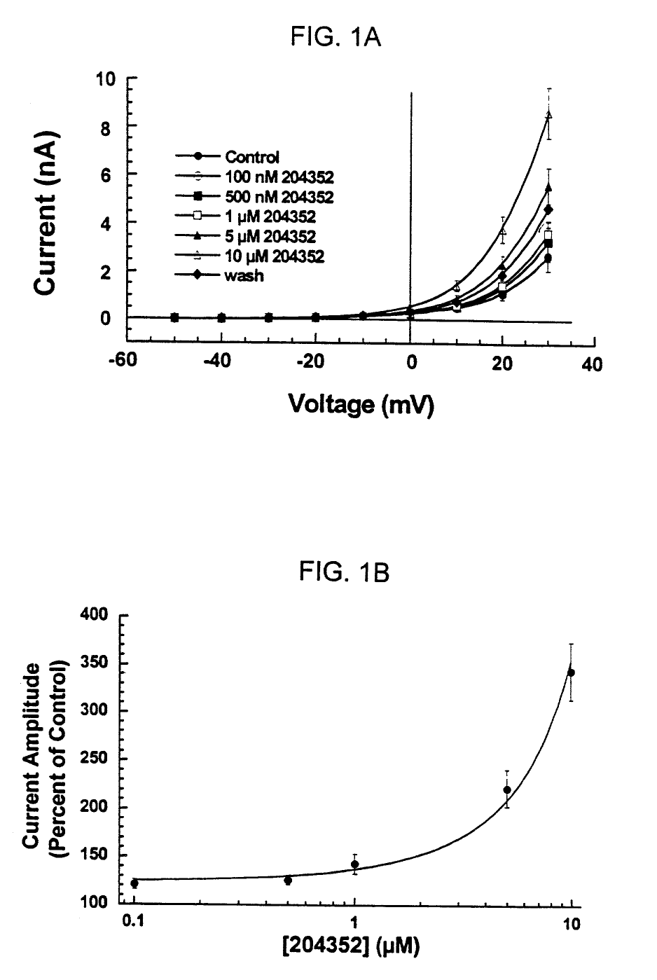

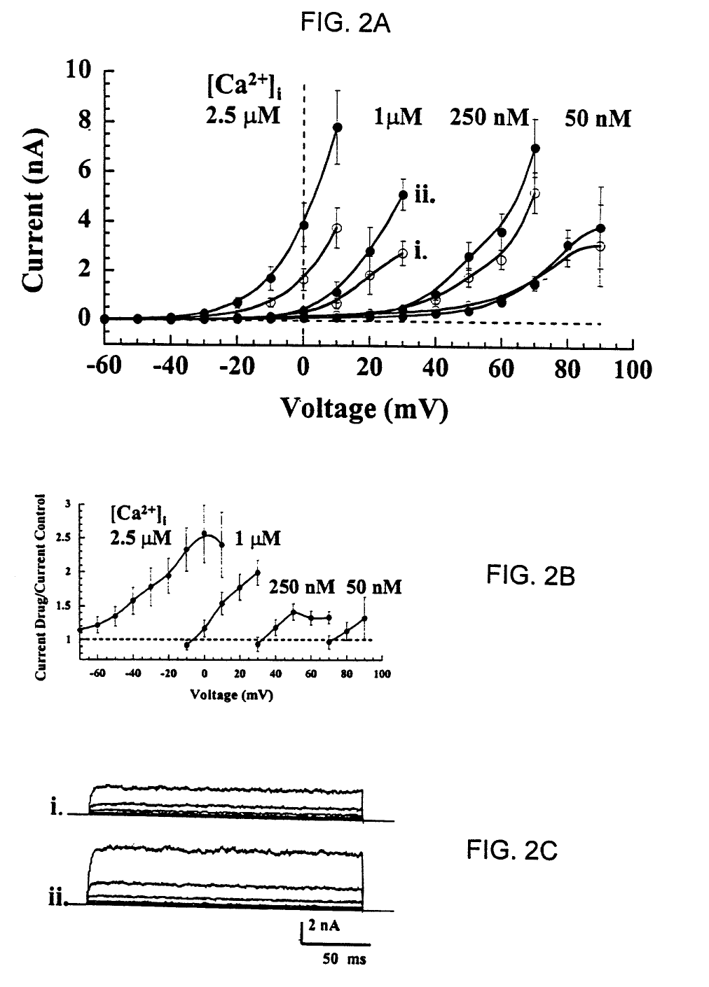

According to the present invention, the BMS-204352 compound has been found to function as a calcium sensitive opener that exerts its action on maxi-K potassium channels in cells having high intracellular calcium concentration ([Ca²⁺]ᵢ), as opposed to cells having low, moderate or normal [Ca²⁺]ᵢ. As described herein, studies using the BMS-204352 compound in accordance with the invention examined the effects of different [Ca²⁺]ᵢ on the ability of the BMS-204352 compound to open maxi-K channels. More specifically, using whole-cell voltage-clamp electrophysiological techniques, hSlo-mediated outward currents were recorded with pipettes containing different concentrations of Ca²⁺ ([Ca²⁺]_{free}=50 nM, 250 nM, 1 μM or 2.5 μM) and clamped cells were exposed to 5 μM of the BMS-204352 compound. (Example 1). The experiments described in Examples 1 and 2 resulted in the BMS-204352 compound increasing whole-cell hSlomediated outward currents in a concentration-dependent and reversible manner (FIGS. 1A and 1B; FIGS. 2A and 2B), in cells having an internal [Ca²⁺]_{free} of 1 μM.

At low intracellular calcium, such as found in “normal” or physiologically normal cells, e.g., [Ca²⁺]ᵢ of about 50 nM to about 250 nM, the BMS-204352 compound was found to have little effect on the open probability of cloned human Slo maxi-K channels. (Example 2). However, as intracellular calcium was increased into the μM range, the BMS-204352 compound became a potent and specific opener of these channels (FIGS. 2A and 2B).

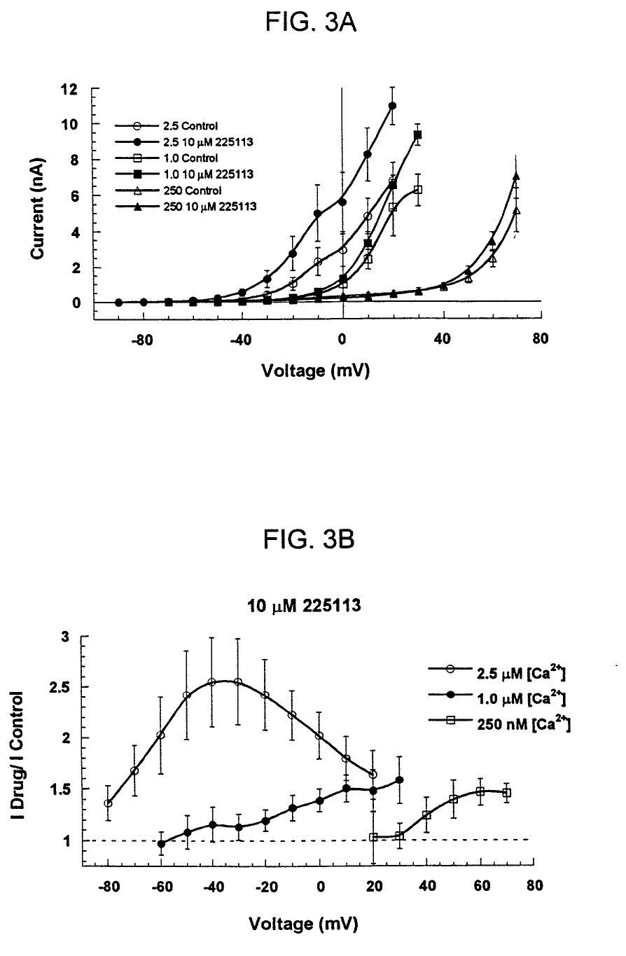

Other novel compounds having the unique high [Ca²⁺]ᵢ-selective and sensitive properties and opener function associated with high [Ca²⁺]ᵢ in accordance with the present invention include the chloro-oxindoles, of which BMS-225113 (i.e., (±)-(5-chloro-2-methoxyphenyl)1,3-dihydro-3-chloro-6-(trifluoromethyl)-2H-indol-2-one) is a particular, yet nonlimiting, example. Like the BMS-204352 compound, the BMS-225113 compound also exerts its effect on cellular maxi-K channels under conditions of high intracellular calcium concentration, while exerting minimal effect on these channels in cells having low or normal intracellular calcium concentration. (FIGS. 3A and 3B and Example 2). In addition, compound BMS-A having the chemical name 3-[(5-chloro-2-hydroxyphenyl)methyl]-5-[4-(trifluoromethyl)phenyl]-1,3,4-oxadiazol-2-(3H)-one, as described in U.S. Pat. No. 5,869,509, has been demonstrated to open cellular maxi-K channels under conditions of high intracellular calcium concentration (Example 12).

The compounds according to the present invention activate maxi-K potassium channels in cells under conditions of high intracellular calcium concentration and do not significantly activate maxi-K potassium channels in cells under low or normal concentrations of intracellular calcium. Such compounds are beneficial in that they are utilizable in diseases and disorders characterized by high intracellular calcium levels as further described herein. Indeed, the compounds and methods according to the present invention provide advantages over other opener compounds in the art as further elucidated below.

Enhancement of maxi-K channel opening during times of excessive cellular stimulation has the potential to afford protection to cells undergoing traumatic stress, and, more particularly, neuroprotection, by attenuating Ca²⁺ entry and reducing Ca²⁺-mediated cellular events, especially those associated with neurodegeneration. The opening of neuronal maxi-K channels in response to cellular depolarization, and increases in [Ca²⁺]ᵢ, constitute an effective endogenous mechanism for repolarizing cells and reducing Ca²⁺ influx. With the exemplified compounds according to the present invention, the most profound drug-induced increase in maxi-K potassium channel opening was observed at micromolar [Ca²⁺]ᵢ, thus indicating that the compounds of this invention can have their greatest effects when cells are exposed to pathogenic levels of excitatory input and increased [Ca²⁺]ᵢ, such as, for example, in conditions of ischemic stroke.

The compounds of this invention stand in contrast to other maxi-K channel openers which have been reported to increase maxi-K channel activity regardless of the concentration of intracellular calcium, such that the activity of other opener compounds is non-selective and is independent of internal cellular calcium concentration. Examples of these types of compounds are described by D. Strbaek et al., 1996, Neuropharmacology, 35:903-914 and M. McKay et al., 1997, J. Neurophysiology, 71:1873. Indeed, the results described in Example 12 herein clearly reveal that while the above-mentioned types of non-selective compounds show a general channel opening activity that is independent of internal calcium concentration, the ability of the compounds of this invention to effect channel opening is highly sensitive to the intracellular concentration of calcium.

Unlike other maxi-K channel openers/modulators that act independently of intracellular calcium concentration, calcium-dependent openers having a selectivity and sensitivity to high concentrations of intracellular calcium, as described herein, significantly affect those channels in cells having high intracellular calcium, e.g., preischemic neuronal cells. Thus, such openers as described by the present invention can be especially useful in treatments or therapies for stroke/ischemia and any other disorder, disease, or condition that is characterized or initiated by high intracellular calcium levels and is amenable and responsive to treatment with an opener of maxi-K potassium channels.

It will also be understood by those having skill in the art that other opener compounds, e.g., small molecule activators of maxi-K channels, which increase the open probability of maxi-K potassium channels in cells which have high intracellular calcium levels, but which do not do so in cells having normal physiological or low intracellular calcium levels, are embraced by the present invention.

Therapeutics/Treatments

In an embodiment of the present invention, the maxi-K opener compounds having sensitivity and selectivity for action on maxi-K channels in cellular environments having a high intracellular calcium concentration may be used for therapeutic purposes. Such opener compounds according to the present invention can be administered in combination with other appropriate therapeutic agents to individuals in need thereof, e.g., patients. Selection of the appropriate agents for use in combination therapy may be made by one of ordinary skill in the art, according to conventional pharmaceutical principles. The combination of therapeutic agents may act synergistically to effect the treatment or prevention of certain diseases and disorders amenable and responsive to treatment by the high [Ca²⁺]ᵢ-sensitive maxi-K opener compounds of this invention, particularly, neurological diseases or disorders, including stroke. Using this approach, one may be able to achieve therapeutic efficacy with lower dosages of each agent, thus reducing the potential for adverse side effects.

Any of the therapeutic methods involving the maxi-K channel openers described herein may be applied to any individual mammal in need of such therapy, including, for example, mammals such as dogs, cats, cows, horses, rabbits, monkeys, and most preferably, humans. Methods of treatment of diseases or disorders characterized by high intracellular calcium levels, e.g., acute ischemic stroke, wherein the maxiK openers having sensitivity and selectivity for opening maxi-K channels in cells with high [Ca²⁺]ᵢ are administered to an individual in need thereof in an amount effective to reduce, ameliorate or alleviate the disease or disorder are encompassed by the present invention. Preferred are methods of providing neuroprotection from ischemic stroke and treatment of neurological diseases or disorders, including, but not limited to, stroke.

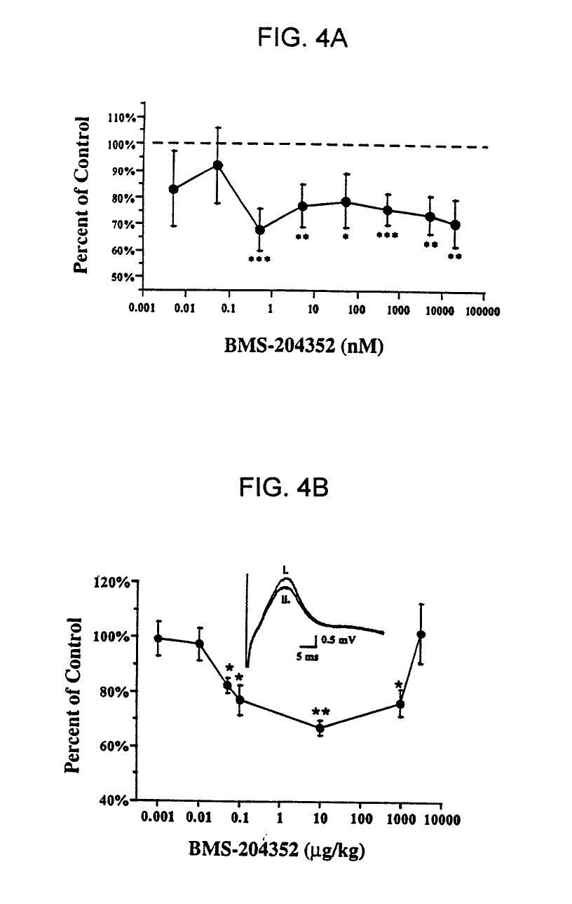

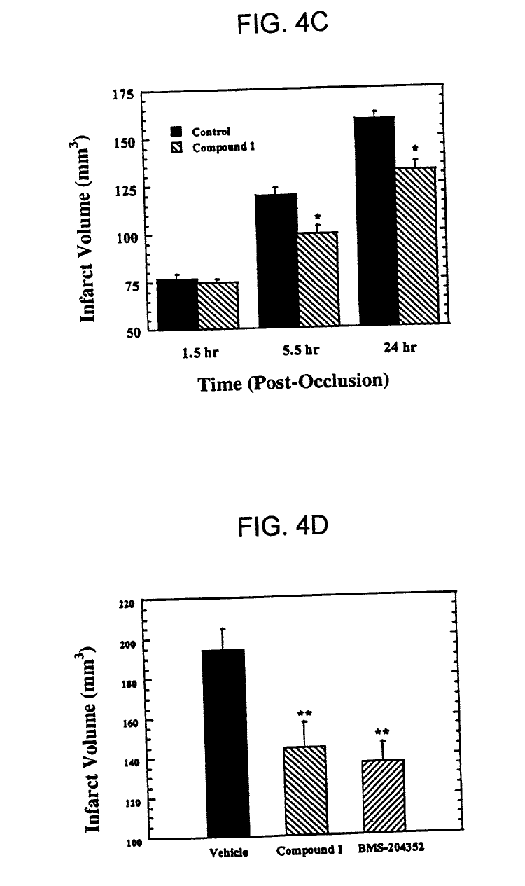

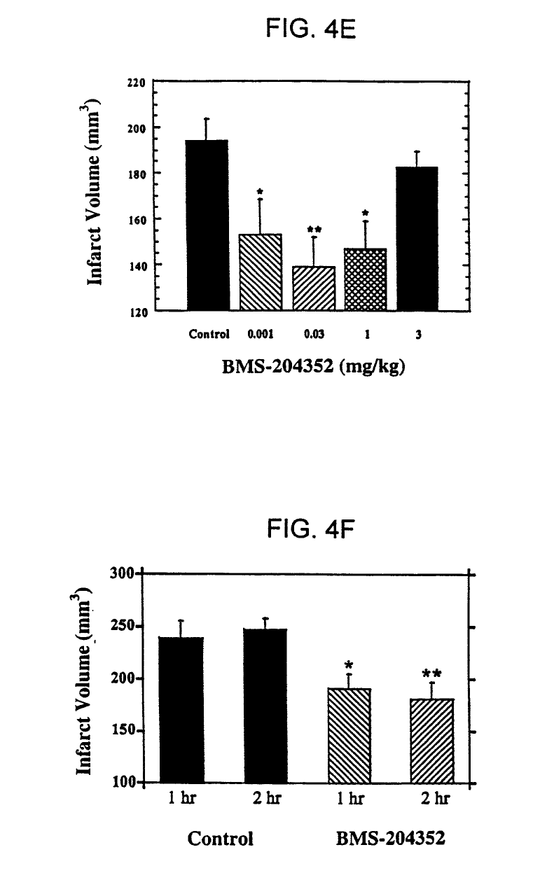

In initial studies performed in rodents to assess in vivo treatment, permanent focal occlusion of the proximal middle cerebral artery (MCA) was combined with delayed drug application, utilizing the BMS-204352 fluoro-oxindole compound, in an effort to recreate conditions of clinical presentation, in which drug is frequently administered at some time after the onset of stroke. Since the neurons to be saved are in a region of reduced blood flow, i.e., the penumbra, the BMS-204352 compound was particularly advantageous because, in addition to all of its other properties, it entered the brain at very high levels. (FIGS. 4A-4F; Example 7).

In another embodiment of the present invention, the maxi-K opener compounds having sensitivity and selectivity for action on maxi-K channels in cellular environments with a high intracellular calcium concentration may be used for preventive treatments, or in pretreatments to prevent the onset of, or reduce the severity of, a disease, disorder, or condition which is characterized or initiated by high intracellular calcium levels and is amenable and responsive to treatment with an opener of maxi-K channels. The administration of the opener compounds according to the present invention for preventive purposes, or as a pretreatment, can be performed in combination with other agents, such as described above for therapeutics.

A nonlimiting example of such pretreatment use is for administration to individuals at risk for, or predisposed to, an above-described disease or disorder, for example, a neurodegenerative disease or disorder, or a chronic neurodegenerative disease or disorder, and more particularly, stroke, global cerebral ischemia, traumatic brain injury, Parkinson's disease, epilepsy, migraine and Alzheimer's disease. In one aspect of this embodiment, the opener compounds according to the present invention can be administered prior to the onset of the disease or disorder, e.g., as a pretreatment for patients who have had one or more prior strokes so as to reduce the risk of, or reduce the severity or degree of, subsequent strokes or related conditions.

Screening Assays and Methods

An embodiment of the present invention involves assays and methods for identifying, detecting, or screening for maxi-K opener compounds that function to open these types of potassium channels in cells having high levels of intracellular calcium, while not having significant opening activity on maxi-K channels in cells having low or normal levels of intracellular calcium. Maxi-K opener compounds determined by the screening and detecting methods of this invention can be used as therapeutics in treatments for diseases and conditions associated with high intracellular calcium concentrations, preferably, neurodegenerative and neurological diseases and disorders, more preferably, acute ischemic stroke.

Candidate agents can be obtained from a wide variety of sources including panels or libraries of synthetic or natural compounds, or from isolated natural compounds, or synthetic compounds or drugs.

One such assay method involves testing a candidate opener compound in a cell-based assay under controlled intracellular calcium conditions. Cells, including primary cells, cell lines, or cell cultures expressing maxi-K channels are employed. Such cells can be naturally-occurring cells which express, or over-express, functional maxi-K channels. Alternatively, the cells can be from established cell lines or cultures that are commercially or otherwise available, (e.g., HEK-293), which have been transfected with cloned maxi-K channel DNA, (e.g., hSlo), and which express maxi-K channels. For cells utilized in the assays for testing for potential maxi-K channel openers according to this invention, conditions are established for cells to have high intracellular calcium concentration [Ca²⁺]ᵢ and for other cells to have low, moderate, or physiologically normal intracellular calcium concentration [Ca²⁺]ᵢ.

Candidate or test compounds are assayed for their ability to function as openers of the maxi-K channels in the presence of high [Ca²⁺]ᵢ, e.g., about 500 nM, or 1, 5, or 10 μM [Ca²⁺]ᵢ, while not having significant opener activity on maxi-K channels at low or normal concentrations of [Ca²⁺]ᵢ, e.g., about 5, 50, 100, or 250 nM [Ca²⁺]ᵢ. For example, standard patch-clamp analyses and recording techniques can be performed, e.g., inside-out and outside-out excised patch, cell-attached patch and whole cell clamp, for example, as described in Examples 1 and 2, to determine if maxi-K channel outward currents are increased or activated after introduction of the candidate or test compound at high [Ca²⁺]ᵢ, compared with the effects of the candidate or test compounds on maxi-K channel outward currents at low or normal preferably [Ca²⁺]ᵢ, preferably, at progressively more negative voltages. If a candidate or test compound is determined to function as a Ca²⁺-sensitive and selective opener of maxi-K channels according to the present invention, it will cause the I-V relationships of the whole-cell currents mediated by the maxi-K channel to shift to the left with increasing intracellular calcium concentration, while having no significant effect under low [Ca²⁺]ᵢ conditions.

If a compound undergoing assay or testing actively opens the maxi-K channels which are expressed by the cells in the assay under conditions of high intracellular calcium concentration, the compound is then also tested in parallel, as described above, to determine its opener function on maxi-K channel-expressing cells, under normal to low intracellular calcium concentrations. A suitable maxi-K opener compound according to this invention is identified or detected via this assay and method if the test compound effectively opens the maxi-K channels in cells under conditions of high [Ca²⁺]ᵢ, but does not effectively open maxi-K channels in cells having normal to low [Ca²⁺]ᵢ.

As a nonlimiting example, the activity of candidate or test opener compounds can be assayed or tested on Xenopus oocytes which have been transduced with cloned maxi-K potassium channels (e.g., hSlo) and which express these channels. In such assays, sufficient hSlo cRNA to produce expression of whole-cell maxi-K currents is injected into, for example, late-stage Xenopus oocytes. Allowing 2 to 4 days for expression, two-electrode voltage-clamp techniques could be employed to record maxi-K channel currents, and the response to drug(s) assessed by examining changes, for example, in the current voltage relationship.

Because oocytes have low endogenous levels of intracellular calcium, a test compound having the properties of the novel maxi-K channel openers as described by the present invention does not significantly open these channels in oocytes, but does open the channels in the maxi-K channel-expressing cells assayed under conditions of high levels of [Ca²⁺]ᵢ as described herein, thereby demonstrating the calcium sensitivity and selectivity of the opener compound. While previously described maxi-K openers were effective at increasing maxi-K current expressed in Xenopus oocytes under endogenous low [Ca²⁺]ᵢ conditions (V. Gribkoff, et al., 1996, Molecular Pharmacology, 50:206-217); the compounds of the present invention, by contrast, do not produce consistent or significant increases in maxi-K current in Xenopus oocytes under these conditions.

In addition, compounds undergoing screening for their ability to specifically target and open maxi-K potassium channels in cells under high [Ca²⁺]ᵢ according to the present invention, while not exerting this action on cells having low to normal [Ca²⁺]ᵢ, can optionally be further assayed for their target specificity against other receptors and enzymes as described in Example 3.

Pharmaceutical Compositions

A further embodiment of the present invention embraces the administration of a pharmaceutical composition, in conjunction with a pharmaceutically acceptable carrier, diluent, or excipient, for any of the above-described therapeutic uses and effects. Such pharmaceutical compositions may comprise one or more opener compounds which are functional and selective in their activity as openers of maxi-K channels in cells having high intracellular calcium concentrations. The compositions may be administered alone or in combination with at least one other agent, such as a stabilizing compound, which may be administered in any sterile, biocompatible pharmaceutical carrier, including, but not limited to, saline, buffered saline, dextrose, organic solvents, e.g., dimethyl sulfoxide (DMSO), ethanol, and water. The compositions may be administered to a patient alone, or in combination with other agents, drugs, hormones, or biological response modifiers.

The pharmaceutical compositions for use in the present invention can be administered by any number of routes including, but not limited to, intravenous, intracranial, intramuscular, intra-arterial, intramedullary, intrathecal, intraventricular, transdermal, subcutaneous, intraperitoneal, intranasal, enteral, topical, sublingual, or oral means.

In addition to the active ingredients (i.e., selective opener compound), the pharmaceutical compositions may contain suitable pharmaceutically acceptable carriers, diluents, or excipients comprising auxiliaries which facilitate processing of the active compounds into preparations which can be used pharmaceutically. Further details on techniques for formulation and administration are provided in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations which can be used orally include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions, e.g., DMSO.

For topical or nasal administration, penetrants or permeation agents that are appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of the opener compounds, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, e.g., using cells expressing or containing maxi-K channel proteins, or in animal models, usually, but not limited to, mice, rats, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, one or more of the novel Ca²⁺-sensitive and selective openers according to the present invention, which ameliorates, reduces, or eliminates the symptoms or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, ED₅₀/LD₅₀. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, sensitivity of the patient, and the route of administration.

The exact dosage will be determined by the practitioner, who will consider the factors related to the individual requiring treatment. Dosage and time of administration are adjusted to provide sufficient levels of the active compound/ingredient or to maintain the desired effect. Factors which may be taken into account include the severity of the individual's disease state, general health of the patient, age, weight, and gender of the patient, diet, time and frequency of administration, drug combination(s), reaction sensitivities, and tolerance/response to therapy. The maxi-K opener compounds can be administered alone or in combination, if necessary or desired.

As a general guide, pharmaceutical compositions for pretreatment or preventive therapies are administered prior to the appearance of the disease or disorder whose severity is being reduced or ameliorated, or whose onset is being prevented or eliminated. It is to be understood that a single pretreatment, or repeated, e.g., successive, pretreatments, with the same or different dose of one or more or the maxi-K channel openers can be used.

In addition, the maxi-K channel opener according to this invention, alone or in combination with another maxi-K channel opener, or with other therapeutic agents, can be administered when a patient presents with disease, for example, immediately upon presentation or at a period of time following onset of the disease. Repeated or successive administration over an appropriate time period can also be employed when a maxi-K channel opener according to the present invention is administered after disease onset. As a non-limiting guide and by way of non-limiting example, the maxi-K channel openers can be administered to a patient in need thereof immediately upon presentation, and/or at a time after disease onset or presentation, e.g., within minutes or hours, and/or up to several hours or days after disease onset or presentation. Long-acting pharmaceutical compositions comprising the maxi-K channel openers of the invention may be administered, for example, every 3 to 4 days, every week, or once every two weeks, depending on the disease or disorder being treated, the half-life and the clearance rate of the particular formulation.

Normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will appreciate that delivery of the compounds according to the invention will be specific to particular cells, conditions, locations, and the like. For example, as described herein, opener compounds that are lipophilic are effective brain penetrants and can be used to affect maxi-K potassium channels in cells affected by brain-related disorders, such as neurons in stroke.

### EXAMPLES

The Examples below are provided to illustrate the subject invention and are not intended to limit the invention.

### Example 1

### Methods

Cell Preparation

HEK-293 cells were transfected with 1-2 μg of pcDNA3 expression plasmid containing hSlo α-subunit cDNA (S. I. Dworetzky et al., 1994, Brain Res. Mol. Brain Res., 27:189-193) using lipofectamine (Gibco) according to the manufacturer's protocol. Cells stably transfected with hSlo were selected in medium supplemented with 0.5 mg/ml Geneticin (G418; Sigma). Transfected HEK-293 cells were grown in Minimum Essential Medium (MEM; Gibco) supplemented with 10% fetal calf serum and G418 at 37° C. in a humidified atmosphere of 5% CO₂ and 95% O₂. Cells were plated on fibronectin-coated glass cover slips in 35 mm culture dishes.

Electrophysiological Recordings

Outward K⁺-mediated currents were examined using standard whole-cell patch-clamp techniques (P. O. Hamill et al., 1981, Pflugers Arch., 391:85-100). Records were filtered at 2 kHz prior to digitization at 10 or 20 kHz. The bathing solution contained the following components: NaCl, 145 mM; KCl, 3 mM; CaCl₂, 2.5 mM; MgCl₂ 1 mM; HEPES, 10 mM; pH 7.4. The recording pipettes were pulled from thick-walled borosilicate glass (2-4 Mω in bathing solution), fire polished and filled with a solution containing: KCl 140 mM; MOPS, 20 mM; EGTA, 1.0 mM; pH 7.2, with CaCl₂ adjusted to produce the required [Ca²⁺]_{free} (determined using the EQCAL program (Biosoft, Cambridge) incorporating the methods of A. Fabiato and F. Fabiato, 1995, J. Physiol., Paris. 75:463-505.

Stimulus generation and data acquisition were controlled by either an Axopatch 200 amplifier or an EPC 9 amplifier, and the Pulse program suite. Data were stored and analyzed off-line. The cell holding potential was −100 mV for experiments with 2.5 μM [Ca²⁺]ᵢ, and −80 or −6 mV for the remaining Ca²⁺ concentrations (1 μM, 250 nM and 50 nM). A series of 10 mV depolarizing voltage steps (100 to 200 ms) appropriate to the [Ca²⁺]ᵢ was applied to evoke a family of outward currents. Series resistance was ≦6Mω before 80% compensation. Currents were measured at steady state near the end of the voltage pulse.

Drugs/Opener Compounds

The BMS-204352 fluoro-oxindole compound and the BMS-225113 chloro-oxindole compound are lipophilic and were therefore made as stock solutions with DMF or DMSO prior to dilution to their final experimental concentrations in bathing solution. If necessary, sonication was used to aid in solubilization. The concentration of solvent was always ≦0.2%. Care was exercised in each experiment to minimize contact of the compounds with surfaces, including glass and plastic, and solutions were freshly prepared before use.

### Example 2

### Patch-Clamp

Using the whole-cell voltage-clamp technique (R. Penner, 1995, “A Practical Guide to Patch Clamping”; In: Single Channel Recording, 2nd Edition, (Eds. B. Sakmann and E. Neher), Plenum Press, New York, pp. 3-30), hSlo-mediated outward currents were recorded with pipettes containing different concentrations of Ca²⁺ (i.e., [Ca²⁺]_{free}=50 nM, 1 μM or 2.5 μM). Clamped cells were exposed to 5 μM of the fluoro-oxindole opener compound BMS-204352, or the chloro-oxindole opener compound BMS-225113, as described in Examples 1.

The results of these experiments showed that the BMS-204352 compound increased whole-cell hSlo-mediated outward currents in a concentration-dependent and reversible manner (FIGS. 1A and 1B). The internal [Ca²⁺]_{free} in these experiments was 1 μM.

For both the BMS-204352 and the BMS-225113 compounds, increasing the intracellular Ca²⁺ concentration by changing the [Ca²⁺]_{free} in the pipette solution caused whole-cell maxi-K currents to activate at progressively more negative voltages. As seen in FIGS. 2A and 3A, increasing [Ca²⁺]ᵢ from 50 nM to 250 nM, 1 μM, or 2.5 μM shifted the current-voltage (I-V) relationships to the left, thus indicating an apparent leftward shift in the half-maximal activation voltage (V_{½}). The addition of the BMS-204352 and BMS-225113 opener compounds (5 μM) increased the hSlo-mediated currents in all four Ca²⁺ concentrations, however the compounds produced significant increases only at the highest concentrations of Ca²⁺ (1 μM and 2.5 μM), (FIGS. 2B and 3B).

These results demonstrate that the BMS-204352 and BMS 225113 compounds are effective and potent openers of human brain maxi-K channel α-subunit (hslo) expressed in HEK-293 cells under conditions of highly elevated intracellular calcium concentration. The I-V relationship of whole-cell currents mediated by hSlo were shifted to the left with increasing [Ca²⁺]ᵢ, thereby suggesting that the half-maximal activation voltage becomes more negative with higher [Ca²⁺]_{free}. Both compounds (5 μM) produced the most profound increase in currents recorded under conditions of 1 and 2.5 μM [Ca²⁺]ᵢ.

In contrast to the above results, in the cell-attached patch-clamp recording configuration with low endogenous intracellular Ca²⁺, the effects of the BMS-204352 compound were relatively modest, and similar modest and inconsistent effects were observed when the compound was applied to Xenopus oocytes expressing hSlo1. The results observed using the novel maxi-K openers of this invention also stand in contrast to the consistent effects of previously described maxi-K channel openers in Xenopus oocytes, for example, under conditions of low endogenous [Ca²⁺]ᵢ (V. Gribkoff, et al., 1996, Molecular Pharmacology, 50:206-217). Both of these systems have low and variable levels of intracellular Ca²⁺.

That the actions of the opener compounds according to this invention are very Ca²⁺-sensitive was confirmed by the experiments and results described in this Example; namely, at low levels of intracellular Ca²⁺ (e.g., 50 nM) the compounds had almost no effect on maxi-K currents, while at higher intracellular Ca²⁺ (e.g., in the micromolar range) the compounds produced progressively greater increases in maxi-K channel-mediated currents, and did so over a wider range of activation voltages (FIGS. 2B, 2C and 3B).

These data propose that the opening of maxi-K channels are significantly augmented at physiologically relevant membrane voltages in the presence of both high intracellular Ca²⁺ and the compound. This can allow for effective control of membrane potential, cell excitability and Ca²⁺ entry in cells having high [Ca²⁺]ᵢ during periods of traumatic stress accompanying a disease state, e.g., in ischemia.

For outside-out and whole cell patch analyses, the solutions used are normal extracellular Na⁺ solution containing (in mM): NaCl, 145; KCl, 3; CaCl₂, 2.5; MgCl₂, 1; HEPES, 10; ±glucose, 10; pH 7.4. Intracellular and extracellular medium for inside-out patches, or pipette solution for whole-cell patch, comprise (in mM): KCl, 140; MOPS, 20; EGTA, 1.0; pH 7.2 CaCl₂ adjusted to produce the required [Ca²⁺]_{free} (determined using the EQCAL program; Biosoft, Cambridge). The cell holding potential for whole-cell recordings is −80 or −60 mV and depolarizing voltage steps appropriate to the internal Ca²⁺ concentration are applied (at high intracellular Ca²⁺ seals become unstable at higher clamp voltages, and are stable at lower voltages for shorter periods).

For outside-out excised patch experiments, patches are bathed in Na⁺ solution. The pipettes (intracellular patch face) contain K⁺ solution with an estimated [Ca²⁺]_{free} of 1 μM, and 200 μM ATPγ-S. Records are filtered at 2 kHz prior to digitization at 5 kHz. The cell holding potential is −60 mV, and 10 mV depolarizing voltage steps from −50 to +130 mV are applied. For inside-out excised patch experiments, the bath and the pipette contain K⁺ solution with [Ca²⁺]_{free}=10 μM. The records are filtered at 2 kHz prior to digitization at 10 kHz. The pipette potential is held at a voltage that allows for the resolution of single channels in the patch prior to drug application. Since more than 1 channel is always present, only visible after drug is applied, the mean open probability, NPₒₚₑₙ, is used to determine he effects of the compounds. In cell-attached patch experiments, the bath and the pipette contains Na⁺ solution. The records are filtered at 2 or 5 kHz prior to digitization at 20 kHz. The pipette potential is held at 0 mV until the initiation of a voltage ramp, or a change to −80 mV for steady-state recordings. NPₒₚₑₙ is used to determine compound effects. Where applicable, conductance (G)/maximal conductance (Gₘₐₓ) vs. voltage (V) relationships are calculated and fit using the Boltzmann function G/Gₘₐₓ=1/(1+e^{(V½-V)/k}), where V_{½} is the membrane potential at half-maximal activation and k is the slope factor. Plots are made using KaleidaGraph software. The EC₅₀ estimate is calculated using a logistic fit of the V_{½} vs. BMS-204352 concentration-response relationship.

### Example 3

### Target Specificity of the Ca²⁺-Sensitive and Selective Maxi-K Channel

### Opener Compounds

To determine the specificity of the effects of the opener compounds, including BMS-204352, on maxi-K potassium channels, the compounds were tested against a representative sample of cloned and native ion channels expressed in Xenopus oocytes and clonal cell lines and were assayed using standard electrophysiological techniques readily known to and practiced by those having skill in the art. These included voltage-dependent potassium channels (Kv1.3, Kv1.5, Kv2.1), Ca²⁺-activated chloride (Cl⁻) channels (native oocyte channels), the cystic fibrosis transmembrane conductance regulator Cl⁻ channel (CFTR) and Ca²⁺ currents (native GH₃ cell Ca²⁺ current). The compounds were found to have no significant effect on Kv currents and Cl⁻ currents, did not activate CFTR (compared with cAMP and the CFTR opener NS004), and did not produce a concentration-dependent effect on native Ca²⁺ currents (15-16% reductions were seen with BMS-204352 at 1 and 10 μM, with similar reductions due to current rundown were observed in control cells).

BMS-204352 was also examined against a binding screen of over 50 receptors and enzyme systems (PanLabs ProfilingScreen® and DiscoveryScreen®). These systems include representatives of most G-protein coupled receptor families, ligand-gated ion channel families, as well as some voltage-gated ion channel binding sites such as the dihydropyridine receptor of L-type Ca²⁺ channels. In an initial test, BMS-204352 produced significant inhibition of the binding of only two ligands, [³H]spiperone, a dopamine D₃ ligand (74% inhibition at 10 μM), and the σ₁ ligand [³H](+)pentazocine (54% inhibition at 10 μM). However, subsequent retest demonstrated no significant interaction with the human dopamine D₃ receptor. The significance of modest inhibition of σ₁ ligand binding is unknown.

### Example 4

### Brain Penetrance of the BMS-204352 Fluoro-oxindole Compound

The ischemic penumbra is an area of low vascular perfusion. For a neuroprotective agent to reach ischemic neurons at therapeutic doses following the onset of a stroke, it should enter the brain at very high levels. Compound BMS-204352 is attractive as a neuroprotective agent due to its high degree of lipophilicity (clogP=5.1). Following intravenous (IV) administration, BMS-204352 quickly enters the rat brain at high levels (Tₘₐₓ=15 min; Cₘₐₓ>10 μg/g in brain following a 5 mg/kg IV bolus dose) with a ratio of brain to plasma of the total compound detected over an 8 hour period equal to 9.6. The plasma half life was 1.6 hour and the brain half life was 1.9 hour. These data demonstrate that BMS-204352 was capable of entering brain quickly at high levels; the high brain to plasma ratio supports the ability of the compound to distribute at effective levels to hypoperfused brain regions such as the ischemic penumbra.

### Example 5

### Safety Profile of the BMS-204352 Fluoro-oxindole Compound

BMS-204352 has an encouraging preclinical safety profile. Administration of BMS-204352 to rats, anesthetized dogs and conscious dogs demonstrated that heart rate and mean arterial blood pressure were unaffected by drug at doses at least 3× higher than the highest neuroprotective dose of 1 mg/kg IV. In single- and multiple-dose studies unaffected by drug at doses at least 3×higher than the highest cytotoxic target organ changes following IV administration for 1 month at doses up to 10 mg/kg/day (rats) and for 10 days at doses up to 20 mg/kg/day (dogs). In addition, compound BMS-204352 was not genotoxic when evaluated in vitro and in vivo, and was not teratogenic in rats and rabbits.

### Example 6

### Opening Maxi-K Channels Attenuated [³H]-Glutamate Release and Synaptic Transmission

Maxi-K channels are localized presynaptically in many brain regions, where they can participate in modulating neurotransmitter release by regulating presynaptic Ca²⁺ entry (H.- G. Knaus et al., 1996, J. Neurosci., 16:955-963). Synaptic transmission is a process initiated by transient high levels of Ca²⁺ influx into terminals. Reduction of synaptic efficacy by openers of the maxi-K potassium channel at glutamatergic synapses of the hippocampus, while expected to be modest in relation to neurotransmitter receptor antagonists, are believed to be a useful neuronal indicator of the presence of a Ca²⁺-sensitive maxi-K channel opener. Field electrical stimulation of hippocampal tissue in vitro in the presence of the compound BMS-204352 resulted in small but significant reductions in the release of radiolabeled glutamate at concentrations between 0.5 nM and 20 μM.

Electrophysiological experiments were also performed in anesthetized rats in vivo. A range of doses were administered to different groups of rats and small, long-lasting, dose-dependent decreases in electrically-evoked hippocampal field potentials in area CA1 were observed at doses between 50 ng/kg and 1.0 mg/kg IV; vehicle as well as lower and higher doses were ineffective. Although these effects were quite modest, they can be used to determine dosing in experiment involving preclinical models.

[³H]-Glutamate Release

Hippocampal tissue (350 μM wedges) from male Sprague-Dawley rats was washed 5 times in Kreb's buffer containing (in mM): NaCl 125; KCl 3.0; MgSO₄ 1.2; CaCl₂ 1.2; NaHCO₃ 22; NaH₂PO₄ 10; glucose 10; 1 unit of adenosine deaminase/mL. Tissue was incubated at 37° C. for 30 minutes in buffer containing 5 μCi of [³H]glutamate (New England Nuclear; Specific activity=50-60 Ci/mMole). Tissue was transferred to superfusion chambers (Brandel Instruments), and washed with buffer for 60 minutes at a flow rate of 1 mL/min. The wash buffer was discarded, and 5 minute fractions were collected thereafter at a flow rate of 0.5 mL/min.

The release of [³H]glutamate was evoked by field electrical stimulation (35 mA, 10 Hz, pulsewidth 2 ms, duration 1 minute). Tissue was stimulated for the first minute of fractions 3 (S₁) and 13 (S₂). The BMS-204352 compound was introduced 30 minutes prior to S₂ (fraction 7) and removed at the end of fraction 15. Radioactivity was quantified using liquid scintillation spectroscopy, and data were expressed as the ratio S₂/S₁ over a range of concentrations of BMS-204352. Statistical analysis consisted of analysis of variance (ANOVA) followed by post-hoc testing (Newmann-Keuls).

Recording of Evoked Potentials in Hippocampus in vivo

Male Long-Evans rats were anesthetized with 1.2 g/kg intraperitoneal (IP) urethane and placed in a stereotaxic apparatus. A stainless steel recording electrode was placed in the CA1 pyramidal cell layer of the hippocampus and a bipolar stimulating electrode was placed in the contralateral hippocampal area CA3. A single electrical stimulus (2-10 V, 0.5 ms) was delivered at a rate of 0.1 pulses/sec. The resulting evoked potential was amplified using a differential amplifier and digitally sampled for off-line analysis. Following a minimum of 1 hour of baseline recording, a vehicle injection was administered, followed 1 hour later by a single dose of the BMS-204352 compound. The compound was solubilized in DMSO, and injections were given IV via jugular catheter. Data were collected for 4-5 hours post-injection. Only one dose of a single compound was tested per animal. At the conclusion of the experiment, animals were perfused and electrode placement was verified histologically. Data were analyzed using repeated measures; ANOVA followed by Kruskal-Wallis tests for significant differences at individual time points as a function of drug concentration.

### Example 7

### Models of Acute Stroke

Permanent focal occlusion of the proximal middle cerebral artery (MCA) is performed using a modified version of the method originally developed by A. Tamura et al. (1981, J. Cerebr. Blood Flow Metab., 1:53-60). In the permanent unilateral MCA, permanent ipsilateral common carotid arteries (CCA) and transient contralateral CCA (PUM,PIC,TCC model), normotensive male Wistar rats are anesthetized with a combination of ketamine (40 mg/kg, i.m.) and xylazine (4.5 mg/kg, i.m.). After exposure of the left MCA via the subtemporal approach, a 2 mm section of the artery is electrocauterized and severed. Within 5 minutes, both CCA are exposed and occluded. The left CCA is permanently occluded by 2 suture ligations, and the right CCA is temporarily occluded for 60 minutes with the use of a micro-aneurysm clip.

The contralateral temporary CCA occlusion reduces collateral flow sufficiently to allow for consistent, large cortical infarcts (S. Brint et al., 1988, Cerebr. Blood Flow Metab., 8:474-485). In experiments with spontaneously hypertensive rats (SHR), an identical MCA occlusion is performed, but with no CCA occlusion. Compounds are dissolved in 2% DMSO/98% propylene glycol, and injected as a single IV bolus in the tail vein at the appropriate time point (usually 2 hours after occlusion). Comparisons are made with groups treated with vehicle alone. Groups contain 12-24 animals. Animals are maintained throughout at 37° C. and monitored by means of a rectal probe. At 24 hours after the occlusion, animals are sacrificed and brains are removed and evaluated by triphenyltetrazolium chloride (TTC) staining to determine the area of infarct. Brain slices are photographed and infarct volumes determined with the assistance of computerized image analysis. Data are analyzed using ANOVAs and appropriate post-hoc tests (Dunnett's).

In MRI MCA occlusion experiments (F. Li et al., 2000, Neurology, 54:689-696), adult male SHR rats (300-330 g) undergo permanent MCA occlusion. During surgery (as described above), the animals are maintained at 37° C. and the rectal temperature is monitored. The animals are then transported to the magnetic resonance laboratory. The rats are anesthetized initially with 3% isoflurane gas anesthesia and later maintained with 1%-1.5% isoflurane gas during MRI measurements. Imaging is performed at 200 MHz using a horizontal 4.7 T magnet fitted with 15 cm gradient coils. Diffusion-weighted images (DWI) are acquired using a STEAM sequence with a single diffusion gradient of 60 mT/m at approximately 1.5 and 5.5 hours post-occlusion.

After the first time point, the vehicle or the opener compound (0.3 mg/kg) dissolved in vehicle, are administered at 2 hours post-occlusion. The DWI images are acquired after the administration of either vehicle or drug at 5.5 hours. T₂-weighted brain images are also obtained at 24 hours after occlusion. Two rats per day are imaged, and MRI findings are confirmed histologically using TTC staining. Statistical analysis is identical to other MCA occlusion experiments.

### Example 8

### Opening of Maxi-K Channels Effectively Reduced Infarct Volume in Rodent Models of Permanent Large-Vessel Occlusion

To examine the activity of the 352 maxi-K channel opener in rodent models of acute focal stroke, the compound was administered intravenously (IV) at 2 hours following the onset of permanent occlusion of the MCA. In one instance, the relative effect of drug administration at 1 or 2 hours post occlusion was compared. Initial characterization of neuroprotective effects was conducted with a racemic compound, Compound 1 (Compound 1: (±)-(5-Chloro-2-methoxyphenyl)-1,3-dihydro-3-fluoro-6-(trifluoromethyl)-2H-indol-2-one). Compound 1 (Compound 1: (±)-(5-Chloro-2-methoxyphenyl)-1,3-dihydro-3-fluoro-6-(trifluoromethyl)-2H-indol-2-one). occlusion was compared using magnetic resonance imaging (MRI) techniques in groups of spontaneously hypertensive rats (SHR). Rats were administered a bolus IV dose of 0.3 mg/kg of compound 1 or vehicle at 2 hours after permanent MCA occlusion onset (FIG. 4A). In this experiment a significant reduction in cortical infarct volume was observed in the compound 1-treated group at 5.5 and 24 hours after occlusion.

In subsequent experiments using SHR rats with permanent MCA occlusion, the BMS-204352 compound produced significant reductions in cortical infarct volume at several doses tested between 0.01 and 0.3 mg/kg. The non-competitive NMDA antagonists MK-801 (Dizocilpine) and CNS-1102 (Cerestat) were ineffective when administered at 2 hours following occlusion onset at doses producing neuroprotection at earlier time points.

In the Wistar normotensive rat, using a model incorporating both permanent unilateral MCA (PUM)/permanent ipsilateral common carotid artery (PIC) occlusion and transient (1 hour) occlusion of the contralateral common carotid artery (TCC) (PUM,PIC,TCC model; see Methods), BMS-204352 and the racemate Compound 1 produced similar levels of neuroprotection (FIG. 4B). The BMS-204352 compound produced significant reductions in cortical infarct volume when administered at doses between 0.001 and 1 mg/kg, but was ineffective at 3 mg/kg (PUM,PIC,TCC model; FIG. 4C). In other experiments, it was determined that the drug was not effective at a dose of 0.001 μg/kg and was confirmed to be ineffective at 3 mg/kg IV, suggesting an ‘inverted-U’ dose-response relationship with a wide effective dose range similar to that seen in the evoked potential experiments (see FIG. 4D). A maximum reduction of 41% was observed with the BMS-204352 compound administered 2 hours post-occlusion, although 20-30% reductions are more typical.

The BMS-204352 compound was equally effective if administered at 1 or 2 hours following occlusion onset (FIG. 4E). These results suggest that opening of maxi-K channels is an effective strategy for reducing the degree of cortical infarct observed following permanent MCA occlusion when administered at least 2 hr following occlusion onset.

In addition, given the characteristics of the maxi-K channel and the novel Ca²⁺-sensitivity of the opener, BMS-204352 is likely to have little effect on the function of non-ischemic cells, and the compound was found to have a preclinical safety profile which would support its evaluation in clinical trials as an effective therapeutic agent for the CNS, specifically targeting neuronal potassium channels.

### Example 9

### (∓)-3-(5-chloro-2-methoxyphenyl)-1,3-dihydro-3-chloro-6-(trifluoromethyl)-2h-indol-2-one

Thionyl chloride (0.613 mL,, 8.4 mmol, 6 equiv) was added to a −78° C. solution of (±)-3-(5-chloro-2-methoxyphenyl)-1,3-dihydro-3-hydroxy-6-(trifluoromethyl)-2H-indol-2-one (0.500 g, 1.4 mmol), [prepared as described in U.S. Pat. No. 5,602,169], and triethylamine (0.726 mL, 8.4 mmol, 6 equiv) in dichloromethane (25 mL). The cold bath was removed and reaction mixture was allowed to warm to room temperature. After 3 hours, the resulting mixture was cooled to 0° C. and carefully quenched by the addition of an aqueous solution of 0.1N hydrochloric acid (10 mL). The contents were poured into water (100 mL) and extracted with ethyl acetate (3×50 ml). The combined organic layer was washed with brine (25 mL), dried (MgSO₄), filtered and concentrated in vacuo. The crude residue was purified using silica gel column chromatography (4/1, hexanes/ethyl acetate) to provided 325 mg (62% yield) of the desired product as an off-white solid. The solid was recrystallized from ethyl acetate/hexane to provide analytically pure title compound.

¹H NMR (400 MHz, DMSO-d₆) 11.24 (1 H, s), 7.91 (1 H, d, J=2.6), 7.52 (1 H, dd, J=8.8, 2.6), 7.30-7.24 (2 H, m), 7.17 (1 H, s), 7.07 (1 H, d, J=8.8), 3.33 (3 H, s); MS ESI 398 (M-H)⁻; Anal. Calc'd for C₁₆H₁₀Cl₂F₃NO₂: C, 51.08; H, 2.67; N, 3.72. Found: C, 51.11; H, 2.53; N, 3.72.

### Example 10

### (3S)-(+)-(5-Chloro-2-methoxyphenyl)-1,3-dihydro-3-chloro-6-(trifluoromethyl)-2H-indol-2-one

The racemic compound of Example 9 may be separated into its enantiomers using a chiral HPLC column such as described in U.S. Pat. No. 5,602,169 to provide the single (+) enantiomer of the title compound of this example.

### Example 11

### (3R)-(−)-(5-Chloro-2-methoxyphenyl)-1,3-dihydro-3-chloro-6-(trifluoromethyl)-2H-indol-2-one

The racemic compound of Example 9 may be separated into its enantiomers using a chiral HPLC column such as described in U.S. Pat. No. 5,602,169 to provide the single (−) enantiomer of the title compound of this example.

### Example 12

### Effects of Intracellular Ca²⁺ Concentration ([Ca²⁺]ᵢ) and BMS-A or NS-1619 on Whole-Cell Currents in HEK-293 Cells Expressing a Human Brain hSlo α-subunit

This example describes experiments that were conducted to determine the effects of intracellular Ca²⁺ concentration ([Ca²⁺]ᵢ) and the use of the BMS-A compound (see U.S. Pat. No. 5,869,509, Example 82), versus a known benzimidazolone compound called NS-1619 [D. Strbaek et al., 1996, Neuropharmacology, 35:903-914 and M. McKay et al., 1997, J. Neurophysiology, 71:1873], on whole-cell currents in HEK-293 cells genetically engineered to express a human brain hSlo α-subunit. For these experiments, the cell holding potential was either −80 or −100 mV and a series of 10 mV depolarizing voltage steps appropriate to the [Ca²⁺]ᵢ, was applied. The standard whole-cell patch clamp technique was used with either [Ca²⁺]ᵢ≡2.5 μM or 50 nM (determined using the EQCAL program, (incorporating the method of A. Fabiato and F. Fabiato, 1975, J. Physiol., Paris. 75: 463-505) in the pipette. The range of voltages used for each [Ca²⁺]ᵢ varied to account for small differences in [Ca²⁺]ᵢ and hSlo expression levels in cells.

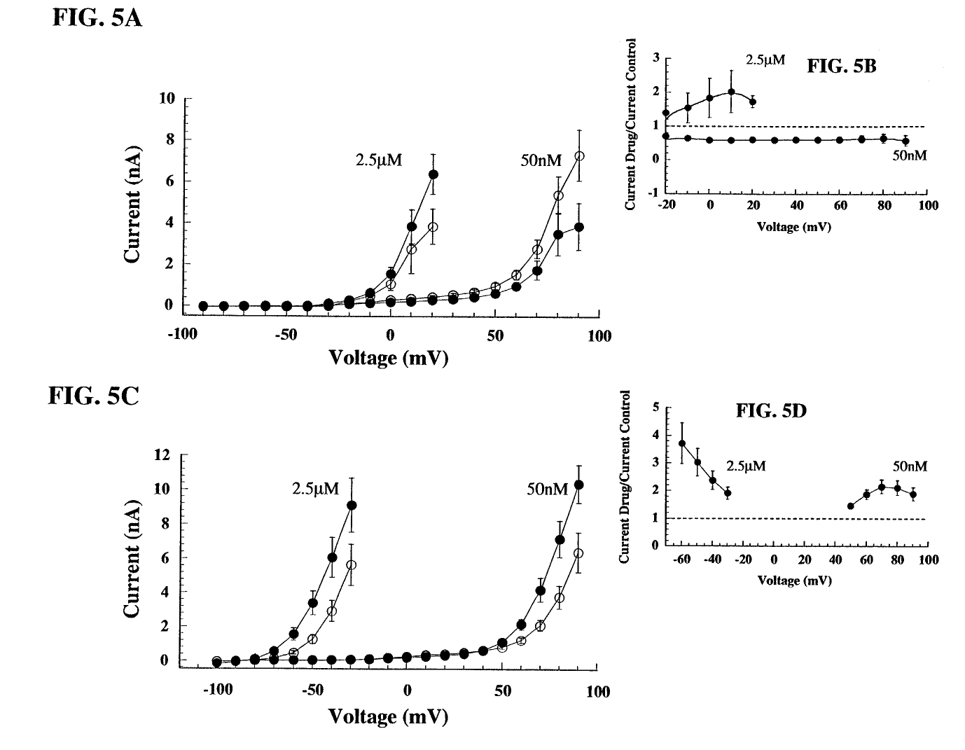

FIG. 5A shows the maxi-K current-voltage (I-V) relationships for control (open symbols) and BMS-A (1 μM) (closed symbols) under two conditions of [Ca²⁺]ᵢ, namely, [Ca²⁺]ᵢ at 50 nM and [Ca²⁺]ᵢ at 2.5 μM. FIG. 5B shows the I-V data with current presented as a ratio (current in drug over control current) versus voltage. Each data point represents the mean±standard error (SE) of 4-8 cells. FIG. 5C shows the maxi-K I-V relationships for experiments with the known benzimidazolone NS-1619 under conditions of the same two [Ca²⁺]ᵢ as used in FIG. 5A. In FIG. 5C, the control is represented by open symbols and drug is shown by closed symbols (circles). FIG. 5D shows the FIG. 5C I-V data with current presented as a ratio versus voltage. Each data point represents the mean ±SE of 9-11 cells. As observed from the results, the high intracellular calcium concentration ([Ca²⁺]ᵢ≡2.5 μM) shifts the I-V relationships to more negative voltages compared with the relationships generated using the low concentration of intracellular calcium ([Ca²⁺]ᵢ≡50 nM), thus indicating an apparent leftward shift in half-maximal activation voltage (Vₘ).

The effect of NS-1619 is the same for each of the [Ca²⁺]ᵢ, increasing the hS/omediated current amplitudes at upper voltages.

In contrast, BMS-A demonstrated a differential effect on channels that was strongly dependent on the [Ca²⁺]ᵢ. In particular, for [Ca²⁺]ᵢ≡2.5 μM, BMS-A increased hSlo-mediated current amplitudes at the highest voltages. For [Ca²⁺]ᵢ≡50 nM, BMS-A actually decreased current amplitudes over a wide range of voltages. These data indicate a general, non-specific channel opening activity for the NS-1619 compound that is independent of [Ca²⁺]ᵢ. However, the ability of BMS-A to effect channel opening is demonstrated to be sensitive to [Ca²⁺]ᵢ.

The contents of all patents, patent applications, published PCT applications and articles, books, references, reference manuals and abstracts as cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. A method of treating a disease or disorder characterized by high intracellular calcium levels in an individual in need thereof, comprising:
providing an effective amount of an opener of maxi-K potassium channels to said individual, wherein said opener activates maxi-K potassium channels in cells under conditions of high intracellular calcium concentration, and does not significantly activate maxi-K potassium channels in cells under low or normal concentrations of intracellular calcium.

2. The method according to claim 1, further wherein influx or introduction of additional calcium into cells having high intracellular calcium concentration is restricted or reduced.

3. The method according to claim 1, wherein the disease or disorder is a neurodegenerative disease or disorder.

4. The method according to claim 3, wherein the neurodegenerative disease or disorder is selected from the group consisting of stroke, global cerebral ischemia, traumatic brain injury, Parkinson's disease, epilepsy, migraine and Alzheimer's disease.

5. The method according to claim 4, wherein the neurodegenerative disease is stroke.

6. The method according to claim 5, wherein the neurodegenerative disease is ischemic stroke or acute ischemic stroke.

7. The method according to claim 6, wherein the cells having a high intracellular calcium concentration are preischemic or ischemic neurons.

8. The method according to claim 1, wherein the maxi-K potassium channel opener is selected from the group consisting of fluoro-oxindole compounds and chloro-oxindole compounds.

9. The method according to claim 8, wherein the fluoro-oxindole compound is selected from the group consisting of (±)-(5-chloro-2-methoxyphenyl)-1,3-dihydro-3-fluoro-6-(trifluoromethyl)-2H-indol-2-one; (3S)-(+)-(5-chloro-2-methoxyphenyl)-1,3-dihydro-3-fluoro-6-(trifluoromethyl)-2H-indole-2-one; and (3S)-(−)-(5-chloro-2-methoxyphenyl)-1,3-dihydro-3-fluoro-6-(trifluoromethyl)-2H-indole-2-one.

10. The method according to claim 9, wherein the fluoro-oxindole compound is (3S)-(+)-(5-chloro-2-methoxyphenyl)-1,3-dihydro-3-fluoro-6-(trifluoromethyl)-2H-indole-2-one.

11. The method according to claim 8, wherein the chloro-oxindole compound is selected from the group consisting of (±)-3-(5-chloro-2-methoxyphenyl)-1,3-dihydro-3-chloro-6-(trifluoromethyl)-2H-indol-2-one; (3S)-(+)-(5-chloro-2-methoxyphenyl)-1,3-dihydro-3-chloro-6-(trifluoromethyl)-2H-indol-2-one; and (3R)-(−)-(5-chloro-2-methoxyphenyl)-1,3-dihydro-3-chloro-6-(trifluoromethyl)-2H-indol-2-one.

12. The method according to claim 1, wherein the maxi-K potassium channel opener is administered prior to or following the onset of the disease or disorder.

13. The method according to claim 5 or claim 6, wherein the maxi-K potassium channel opener is administered prior to or following the onset of stroke, ischemic stroke, or acute ischemic stroke.

14. A method of treating stroke in an individual in need thereof, comprising:
administering to the individual an effective amount of a maxi-K channel opener, said opener having opener activity on maxi-K potassium channel proteins in neuronal cells having a high intracellular calcium concentration, while having no significant opener activity on maxi-K potassium channel proteins in neuronal cells having normal or low intracellular calcium concentration.

15. The method according to claim 14, wherein the maxi-K channel opener is selected from the group consisting of fluoro-oxindole compounds and chloro-oxindole compounds.

16. The method according to claim 15, wherein the fluoro-oxindole compound is selected from the group consisting of (±)-(5-chloro-2-methoxyphenyl)-1,3-dihydro-3-fluoro-6-(trifluoromethyl)-2H-indol-2-one; (3S)-(+)-(5-chloro-2-methoxyphenyl)-1,3-dihydro-3-fluoro-6-(trifluoromethyl)-2H-indole-2-one; and (3S)-(−)-(5-chloro-2-methoxyphenyl)-1,3-dihydro-3-fluoro-6-(trifluoromethyl)-2H-indole-2-one.

17. The method according to claim 16, wherein the fluoro-oxindole compound is (3S)-(+)-(5-chloro-2-methoxyphenyl)-1,3-dihydro-3-fluoro-6-(trifluoromethyl)-2H-indole-2-one.

18. The method according to claim 15, wherein the chloro-oxindole compound is selected from the group consisting of (±)-3-(5-chloro-2-methoxyphenyl)-1,3-dihydro-3-chloro-6-(trifluoromethyl)-2H-indol-2-one; compound is selected from the group consisting of (±)-3-(5-chloro-2-methoxyphenyl)-1,3-dihydro-3-chloro-6-(trifluoromethyl)-2H-indol-2-one; (3S)-(+)-(5-chloro-2-methoxyphenyl)-1,3-dihydro-3-chloro-6-(trifluoromethyl)-2H-indol-2-one; and (3R)-(−)-(5-chloro-2-methoxyphenyl)-1,3-dihydro-3-chloro-6-(trifluoromethyl)-2H-indol-2-one. acute ischemic stroke is treated.

19. The method according to claim 14, wherein ischemic stroke or acute ischemic stroke is treated.

20. The method according to claim 14 or claim 19, wherein the maxi-K channel opener is administered prior to or following the onset of stroke, ischemic stroke or acute ischemic stroke.

21. The method according to claim 14, wherein the maxi-K channel opener provides cortical neuroprotection by restricting entry of calcium into neuronal cells exposed to toxic levels of calcium.

22. A method of treating a disease or disorder characterized by high intracellular calcium levels in an individual in need thereof, comprising:
a) providing to the individual an opener of maxi-K channels wherein the opener is sensitive to high intracellular calcium concentration and targets maxi-K channels in cells associated with the disease or disorder and having high intracellular calcium concentration, while not significantly targeting cells having low or normal intracellular calcium concentration; and
b) reducing or restricting influx of additional calcium into the cells associated with the disease or disorder, increasing potassium efflux and regulating membrane potential, thereby protecting the cells associated with the disease or disorder from toxicity or death.

23. The method according to claim 22, wherein the maxi-K channel opener is a fluoro-oxindole compound or a chloro-oxindole compound.

24. The method according to claim 23, wherein the fluoro-oxindole compound is selected from the group consisting of (±)-(5-chloro-2-methoxyphenyl)-1,3-dihydro-3-fluoro-6-(trifluoromethyl)-2H-indol-2-one; (3S)-(+)-(5-chloro-2-methoxyphenyl)-1,3-dihydro-3-fluoro-6-(trifluoromethyl)-2H-indole-2-one; and (3S)-(−)-(5-chloro-2-methoxyphenyl)-1,3-dihydro-3-fluoro-6-(trifluoromethyl)-2H-indole-2-one.

25. The method according to claim 24, wherein the fluoro-oxindole compound is (3S)-(+)-(5-chloro-2-methoxyphenyl)-1,3-dihydro-3-fluoro-6-(trifluoromethyl)-2H-indole-2-one.

26. The method according to claim 24, wherein the fluoro-oxindole compound is (3S)-(−)-(5-chloro-2-methoxyphenyl)-1,3-dihydro-3-fluoro-6-(trifluoromethyl)-2H-indole-2-one.

27. The method according to claim 23, wherein the chloro-oxindole compound is selected from the group consisting of (±)-3-(5-chloro-2-methoxyphenyl)-1,3-dihydro-3-chloro-6-(trifluoromethyl)-2H-indol-2-one; (3S)-(+)-(5-chloro-2-methoxyphenyl)-1,3-dihydro-3-chloro-6-(trifluoromethyl)-2H-indol-2-one; and (3R)-(−)-(5-chloro-2-methoxyphenyl)-1,3-dihydro-3-chloro-6-(trifluoromethyl)-2H-indol-2-one. and (3R)-(−)-(5-chloro-2-methoxyphenyl)-1,3-dihydro-3-chloro-6-(trifluoromethyl)-2H-indol-2-one.

28. The method according to claim 22, wherein the disease or disorder is a neurogenerative disease or disorder.

29. The method according to claim 28, wherein the neurodegenerative disease or disorder is selected from the group consisting of stroke, global cerebral ischemia, traumatic brain injury, Parkinson's disease, epilepsy, migraine and Alzheimer's disease.

30. The method according to claim 29, wherein the neurodegenerative disease or disorder is stroke.

31. The method according to claim 30, wherein the neurodegenerative disease or disorder is ischemic stroke or acute ischemic stroke.

32. The method according to claim 22, wherein the cells associated with the disease or disorder and having a high intracellular calcium concentration are at-risk preischemic neurons or ischemic neurons.

33. The method according to claim 29, wherein the neurodegenerative disease or disorder is traumatic brain injury.

34. The method according to claim 22, wherein the maxi-K channel opener is administered prior to or after the onset of the disease or disorder.

35. A method of providing neuroprotection from stroke in an individual in need thereof, comprising: administering an effective amount of a maxi-K potassium channel opener compound that activates maxi-K potassium channel proteins in neurons having a high intracellular calcium concentration, while having no significant opener activity on maxi-K potassium channel proteins in neurons having low or normal intracellular calcium concentration.

36. A method of providing neuroprotection from stroke in an individual in need thereof, comprising: administering an effective amount of a fluoro-oxindole or chloro-oxindole compound to the individual wherein the compound is a maxi-K potassium channel opener compound that activates maxi-K potassium channel proteins in neurons having a high intracellular calcium concentration, while having no significant opener activity on maxi-K potassium channel proteins in neurons having low or normal intracellular calcium concentration, thereby providing cortical neuroprotection by restricting entry of calcium into the neurons at risk for neurotoxicity or death.

37. The method according to claim 35 or claim 36, wherein the maxi-K channel opener compound is a fluoro-oxindole compound.

38. The method according to claim 37, wherein the fluoro-oxindole compound is selected from the group consisting of (±)-(5-chloro-2-methoxyphenyl)-1,3-dihydro-3-fluoro-6-(trifluoromethyl)-2H-indol-2-one; (3S)-(+)-(5-chloro-2-methoxyphenyl)-1,3-dihydro-3-fluoro-6-(trifluoromethyl)-2H-indole-2-one; and (3S)-(−)-(5-chloro-2-methoxyphenyl)-1,3-dihydro-3-fluoro-6-(trifluoromethyl)-2H-indole-2-one.

39. The method according to claim 38, wherein the fluoro-oxindole compound is (3S)-(+)-(5-chloro-2-methoxyphenyl)-1,3-dihydro-3-fluoro-6-(trifluoromethyl)-2H-indole-2-one.

40. The method according to claim 38, wherein the fluoro-oxindole compound is (3S)-(−)-(5-chloro-2-methoxyphenyl)-1,3-dihydro-3-fluoro-6-(trifluoromethyl)-2H-indole-2-one.

41. The method according to claim 35 or claim 36, wherein the maxi-K channel opener compound is a chloro-oxindole compound.

42. The method according to claim 41, wherein the chloro-oxindole compound is selected from the group consisting of (±)-3-(5-chloro-2-methoxyphenyl)-1,3-dihydro-3-chloro-6-(trifluoromethyl)-2H-indol-2-one; (3S)-(+)-(5-chloro-2-methoxyphenyl)-1,3-dihydro-3-chloro-6-(trifluoromethyl)-2H-indol-2-one; and (3R)-(−)-(5-chloro-2-methoxyphenyl)-1,3-dihydro-3-chloro-6-(trifluoromethyl)-2H-indol-2-one.

43. The method according to claim 35 or claim 36, wherein the maxi-K channel opener compound is administered prior to or after the onset of stroke.

44. The method according to claim 35 or claim 36, wherein the neuroprotection is for ischemic stroke or acute ischemic stroke.

45. The method according to claim 35 or claim 36, wherein the neurons having high intracellular calcium concentration are preischemic and/or ischemic neurons.

46. A method of screening for or identifying opener or agonist compounds capable of activating maxi-K potassium channel proteins under conditions of high intracellular calcium concentration, while not significantly activating maxi-K channel proteins under conditions of normal physiological or low intracellular calcium concentration comprising:
a) contacting a test compound with a maxi-K potassium channel protein-expressing cell under conditions of high intracellular calcium concentration; and
b) selecting as said opener or agonist compounds those test compounds which activate maxi-K potassium channel proteins in the cells under conditions of high intracellular calcium concentration, and which do not significantly activate maxi-K potassium channel proteins in cells under low to normal physiological intracellular calcium concentration.

47. The method according to claim 46, wherein the cells having high intracellular calcium concentration are mammalian cells transfected with cloned maxi-K potassium channel protein-encoding DNA, wherein the maxi-K channel protein is expressed.

48. The method according to claim 46, wherein conditions of high intracellular calcium concentration comprise a calcium concentration in the range of greater than about 250 nanomolar to about 10 micromolar [Ca²⁺]ᵢ.

49. The method according to claim 46, wherein the cells under low or normal physiological intracellular calcium concentration are Xenopus oocytes transfected with maxi-K channel protein-encoding DNA, wherein the transfected oocytes express the maxi-K channel protein.

50. The method according to claim 46, wherein conditions of low to normal physiological intracellular calcium concentration comprise a calcium concentration in the range of about 5 nanomolar to 250 nanomolar [Ca²⁺]ᵢ.

51. The method according to claim 46, wherein activation of the maxi-K channels in cells by the test compound is determined using patch-clamp analysis.

52. The method according to claim 51, wherein the patch-clamp analysis is selected from the group consisting of inside-out excised patch, outside-out excised patch, cell-attached excised patch and whole-cell clamp patch.

53. The method according to claim 52, wherein the patch-clamp analysis is whole-cell clamp patch.

54. The method according to claim 46, wherein activation of maxi-K channel proteins comprises increase or activation of maxi-K channel outward currents after introduction of the test compound.

55. A method of assaying for compounds to identify calcium-sensitive opener compounds which selectively activate maxi-K potassium channel proteins under conditions of high intracellular calcium concentration and which do not significantly activate maxi-K potassium channel proteins under conditions of low intracellular calcium concentration or normal intracellular calcium concentration, comprising:
(a) contacting a maxi-K potassium channel protein with a test compound under conditions of high intracellular calcium concentration; and
(b) determining if the opener compound selectively activates the maxi-K potassium channel protein in the presence of high intracellular calcium concentration, and does not significantly activate the maxi-K potassium channel protein in the presence of low or physiologically normal intracellular calcium concentration.

56. The method according to claim 55, further comprising a positive control which is a maxi-K potassium channel opener compound having the characteristics of being sensitive to high intracellular calcium concentration and selectively targeting cellular maxi-K potassium channels under conditions of high intracellular calcium concentration.

57. The method according to claim 56, wherein the positive control is a fluoro-oxindole or a chloro-oxindole maxi-K potassium channel opener compound.

58. The method according to claim 57, wherein the positive control is a fluoro-oxindole maxi-K potassium channel opener compound selected from the group consisting of (±)-(5-chloro-2-methoxyphenyl)-1,3-dihydro-3-fluoro-6-(trifluoromethyl)-2H-indol-2-one; (3S)-(+)-(5-chloro-2-methoxyphenyl)-1,3-dihydro-3-fluoro-6-(trifluoromethyl)-2H-indole-2-one; and (3S)-(−)-(5-chloro-2-methoxyphenyl)-1,3-dihydro-3-fluoro-6-(trifluoromethyl)-2H-indole-2-one.

59. The method according to claim 57, wherein the positive control is a chloro-oxindole maxi-K potassium channel opener compound selected from the group consisting of (±)-3-(5-chloro-2-methoxyphenyl)-1,3-dihydro-3-chloro-6-(trifluoromethyl)-2H-indol-2-one; (3S)-(+)-(5-chloro-2-methoxyphenyl)-1,3-dihydro-3-chloro-6-(trifluoromethyl)-2H-indol-2-one; and (3R)-(−)-(5-chloro-2-methoxyphenyl)-1,3-dihydro-3-chloro-6-(trifluoromethyl)-2H-indol-2-one.

60. A maxi-K opener compound selected or identified according to the method of claim 46 or claim 55.

61. A maxi-K potassium channel opener compound characterized by opening or activating maxi-K potassium channels in cells under conditions of high intracellular calcium concentration, and not significantly opening or activating maxi-K potassium channels in cells under low or normal concentrations of intracellular calcium, wherein said maxi-K opener compound reduces or restricts influx or introduction of additional calcium into cells under conditions of high intracellular calcium concentration.

62. A compound which is (±)-3-(5-chloro-2-methoxyphenyl)-1,3-dihydro-3-chloro-6-(trifluoromethyl)-2H-indol-2-one.

63. A compound which is (3S)-(+)-(5-chloro-2-methoxyphenyl)-1,3-dihydro-3-chloro-6-(trifluoromethyl)-2H-indol-2-one.

64. A compound which is (3R)-(−)-(5-chloro-2-methoxyphenyl)-1,3-dihydro-3-chloro-6-(trifluoromethyl)-2H-indol-2-one.

65. A pharmaceutical composition comprising the compound according to any one of claims 62 to 64, and a physiologically acceptable diluent, carrier, or excipient.
